(19) United States
(12) Patent Application Publication
(10) Pub. No.: US 2019/0336511 A1
(43) Pub. Date: Nov. 7, 2019

(54) Treatment for Inflammation and Pain with Reduced Gastro-Intestinal Side Effects Combining Methylprednisolone, Paracetamol, and Lansoprazole

(71) Applicant: Georgiy Brusovanik, Miami, FL (US)

(72) Inventor: Georgiy Brusovanik, Miami, FL (US)

(21) Appl. No.: 16/402,179

(22) Filed: May 2, 2019

Related U.S. Application Data
(60) Provisional application No. 62/665,510, filed on May 2, 2018.

(51) Int. Cl. A61K 31/573; A61K 31/167; A61K 31/4439; A61P 1/04; A61P 29/00

(52) U.S. Cl. CPC A61K 31/573; A61K 31/167; A61P 29/00; A61P 1/04; A61K 31/4439

(57) ABSTRACT

A multimodal treatment for inflammation and pain with reduced gastro-intestinal side effects combines an anti-inflammatory, an analgesic, and a composition for reducing stomach acid. A preferred composition utilizes a steroid as the anti-inflammatory and a proton inhibitor as the composition for reducing stomach acid. A composition including methylprednisolone as the anti-inflammatory, paracetamol as the analgesic, and esomeprazole or lansoprazole as the composition for reducing stomach acid is particularly effective. The composition can be combined into a single pill for more convenient and controlled delivery. The treatment can be used to treat joint pain, particularly back and neck pain.

### CROSS-REFERENCE TO RELATED APPLICATIONS

This application claims the benefit of U.S. Provisional Application No. 62/665,510, filed May 2, 2018, which is hereby incorporated by reference.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The invention relates to medications for back and neck pain.

### Description of the Related Art

Back and neck pain are common and account for a large percentage of both emergency room and primary care visits. A typical treatment for back and neck pain combines an analgesic and a steroidal medication as a first- or second-line treatment.

Steroids have been utilized in a wide range of treatments and have existed long enough that their safety and side effects have become well known. The side effect profile of short-term steroid use is low. Steroids have such an established safety record that they are widely prescribed to treat asthma in children.

Methylprednisolone is a corticosteroid medication used to suppress the immune system and decrease inflammation. Methylprednisolone is a common first-line treatment for back and neck pain. Methylprednisolone is sold under the trademark MEDROL®. Packaged courses of treatment using methylprednisolone are sold under the trade name MEDROL PAK™. A typical six-day course of treatment of methylprednisolone provides a series of tapered doses. The price of methylprednisolone in 2018 is about $15.96 in consumer pharmacies and it is widely available in most pharmacies. The side-effect profile of methylprednisolone is low. However, gastro-intestinal (GI) side-effects are possible and are a source of concern for the prescribing provider.

In the treatment of back and neck pain, analgesics are typically used. A commonly prescribed analgesic for back and neck pain is paracetamol.

Paracetamol, also known as acetaminophen or APAP, is a medicine used to treat pain and fever. Paracetamol is sold under the trademark TYLENOL®. Paracetamol is typically used for mild to moderate pain relief. Paracetamol is often sold in combination with other medications, such as in many cold medications.

Addition of paracetamol to narcotics is an established and long-standing practice and is a significant factor in “multimodal pain treatment”.

The following studies investigated the effects of combining methylprednisolone and paracetamol.
Smith, “Potential Analgesic Mechanisms of Acetaminophen,” Pain Physician, 2009 January-February; 12(1):269-80.
Beaver, W T, “Aspirin and Acetaminophen as Constituents of Analgesic Combinations,” Arch Intern Med. 1981; 141 (3, Spec No.): 293-300.
Beaver W T, “Combination analgesics,” Am J Med. 1984; 10 (3A):38-53.
Raffa et al. “Oxycodone Combinations for Pain Relief,” Drugs Today. 2010 June; 46(6): 379-398.

Taking more than a recommended daily dosage of paracetamol can cause liver damage. Patients with chronic back and neck pain are particularly at risk of paracetamol overdoses because the patients typically take high doses of paracetamol when having back and neck pain. Then as a result of taking high dosages throughout the day, the patient exceeds the recommended daily dosage and risks liver damage.

Protein Pump Inhibitors (PPIs) reduce the production of acid by the stomach. PPIs have an established history of lowering gastro-intestinal side effects when combined with non-steroidal anti-inflammatory medications.

Esomeprazole is an example of a protein pump inhibitor. Esomeprazole has an established history of lowering (GI) side effects when combined with non-steroidal anti-inflammatory medications. Esomeprazole is sold under the trademark NEXIUM®. Applications of esomeprazole are described in an article by Choi et al. titled, “Comparison of the pharmacokinetics and tolerability of HCP1004 (a fixed-dose combination of naproxen and esomeprazole strontium) and VIMOVO® (a marketed fixed-dose combination of naproxen and esomeprazole magnesium) in healthy volunteers” in the Jul. 31, 2015 issue of Drug Des. Devel. Ther. pp 4127-35.

### BRIEF SUMMARY OF THE INVENTION

An object of the invention is to provide a multimodal treatment for pain and inflammation with reduced gastro-intestinal side effects. The multimodal treatment is a composition that includes an analgesic and a steroid for the treatment of pain and inflammation and adds a compound for reducing stomach acid.

A further object of the invention is to simplify the administration of a multimodal treatment for pain and inflammation with reduced gastro-intestinal side effects by providing a treatment given in a single dose. In this application, a “single dose” means that the multimodal treatment is given in one measured quantity of therapeutic agent: for example, as one pill, one liquid volume, or one injection. A signal dose contrasts a multi dose. In a multi dose, more than one measured quantity, each with a different therapeutic agent is delivered at one time: for example, at noon, a patient takes a first pill with a dose of an analgesic, a second pill with a dose of a steroid, and a third pill with a protein-pump inhibitor. The term “single dose” is not meant to preclude a course of treatment over which a series of single doses are delivered periodically to a patient. So, in other words, the treatment can include a periodic course of single doses taken over a period of time to prevent recurrence of the pain and inflammation.

A further object of the invention is to provide a treatment in which the amounts and delivery rates of each of the therapeutic composition are adjusted so the therapeutic effects of the components allow for the safe and effective consumption of a subsequent dose that includes all the components.

A further object of the invention is to provide a multimodal treatment for inflammation and pain of joints, and particularly back and neck pain with reduced gastro-intestinal side effects compared to the gastro-intestinal side effects of any analgesic and anti-inflammatory components without any additional components.

With the foregoing and objects in view there is provided, a treatment (i.e. a composition) that includes a therapeutically-effective amount of steroid anti-inflammatory composition, a therapeutically-effective amount of an analgesic, and a therapeutically-effective amount of protein pump inhibitor.

Examples of suitable steroid anti-inflammatory compositions include methylprednisolone, cortisone, hydrocortisone, and prednisone.

Examples of suitable analgesic compositions include paracetamol, ibuprofen, naproxen, sulindac, ketoprofen, tolmetin, todolac, fenoprofen, diclofenac, flurbiprofen, misoprostol, piroxicam, indomethacin, esomeprazole, meloxicam, nabumetone, and oxaprozin.

Examples of suitable protein pump inhibitors include esomeprazole, lansoprazole, dexlansoprazole, pantoprazole, rabeprazole, and omeprazole.

With the foregoing and other objects in view there is provided, in accordance with the invention, a treatment (i.e. a composition) that includes therapeutically effective amounts of methylprednisolone, paracetamol, and esomeprazole. The methylprednisolone is an anti-inflammatory. Paracetamol is an analgesic. When used together, methylprednisolone and paracetamol provide improve treatment of inflammation and pain in patients with back and neck pain than, patients treated with methylprednisolone or paracetamol alone. Esomeprazole and lansoprazole are proton pump inhibitors that decreases the gastro-intestinal side effects of methylprednisolone without decreasing the effectiveness of the methylprednisolone and paracetamol.

Therapeutic amounts of methylprednisolone range from two to thirty-two milligrams (2-32 mg) per dose. The amount of methylprednisolone in a given dose of a course of treatment can vary compared to the amount in other doses. For example, a first dosage can include a greater amount of methylprednisolone than later doses. The amount of methylprednisolone per single dose can be tapered during the second week of treatment.

Therapeutic amounts of paracetamol range from one hundred to one thousand three hundred milligrams (325-1,300 mg) per dose.

Therapeutic amounts of lansoprazole range from two to thirty-two milligrams (2-32 mg) per dose.

To simplify the treatment for patients, pharmacists, and care givers, the methylprednisolone, paracetamol, and esomeprazole are combined into a mixture, for example a single pill containing methylprednisolone, paracetamol, and esomeprazole.

The treatment can be provided as a course of doses. The particular doses may be different and are to be given at a particular time during the treatment. For example, the amount of methylprednisolone can taper in the doses over the course of treatment. In addition, the amount of esomeprazole can be tapered as the amount of methylprednisolone tapers.

While risks of GI side effects from methylprednisolone are feared, a true study of the incidences of side-effects is unknown. Methylprednisolone is prescribed in the emergency rooms, urgent care centers, and primary care settings but the true frequency of prescriptions is not clear. Searches have found no known studies on contraindications between paracetamol, esomeprazole, and methylprednisolone. However, there are studies of each of these medications separately and the safety profile is very reasonable.

A further object of the invention is to provide a treatment that can utilize prior FDA approvals to allow for an exemption to the FDA's normal prohibition to new treatments. Although, the combination of paracetamol, esomeprazole, and methylprednisolone into a single treatment has beneficial therapeutic, patient-compliance, and commercial effects, because the FDA previously approved paracetamol, esomeprazole, and methylprednisolone, separately, an exemption obviating normally expensive FDA approvals can be requested.

An object of the invention is providing a treatment in which the amount of methylprednisolone in the composition produces a stable, reproducible compound with a predictable half-life in a titrated dose, like the current way that methylprednisolone is separately prescribed. A further object of the invention is to provide a treatment in which the amount of methylprednisolone in the doses in a course of treatment is like the amount of methylprednisolone in the courses of treatment sold under the trade name MEDROL PAK™.

The invention further encompasses a method of treating inflammation and pain multimodially that reduces gastro-intestinal side effects. The method calls for providing a dose that combines a therapeutically effective amount of methylprednisolone, a therapeutically-effect amount of paracetamol, and a therapeutically effective amount of esomeprazole.

The method of treatment can include the step of adjusting the amounts and delivery rates of the methylprednisolone, the paracetamol, and the esomeprazole so that a subsequent dose combining methylprednisolone, paracetamol, and esomeprazole can be safely and effectively given.

The method of treatment can include a course of doses in which the doses are scheduled at particular times and with particular relative amounts of methylprednisolone, paracetamol, and esomeprazole that the pain and inflammation is being treated safely and effectively throughout the course.

In accordance with the objects of the invention a treatment (i.e. a composition) in a single-dose ingestible pill that includes therapeutically effective amounts of a steroidal anti-inflammatory and a non-steroidal analgesic.

In accordance with the objects of the invention a treatment (i.e. a composition) in a single-dose ingestible pill that includes therapeutically effective amounts of a steroidal anti-inflammatory and a proton pump inhibitor.

In accordance with the objects of the invention a treatment (i.e. a composition) in a single-dose ingestible pill that includes a therapeutically effective amounts of a non-steroidal analgesic and a proton pump inhibitor.

Other features that are considered as characteristic for the invention are set forth in the appended claims.

Although the invention is described herein as embodied in a treatment for inflammation and pain with reduced gastro-intestinal side effects combining methylprednisolone, paracetamol, and esomeprazole, the invention should not be limited to the details shown in those embodiments because various modifications and structural changes may be made without departing from the spirit of the invention while remaining within the scope and range of equivalents of the claims.

### DETAILED DESCRIPTION OF THE INVENTION

A preferred embodiment of a treatment for inflammation and pain has the following composition:

100-600 mg Paracetamol;

4-24 mg Lansoprazole; and

4-24 mg Methylprednisolone.

A preferred delivery system of the treatment is to provide a single dose pill that includes the paracetamol, the lansoprazole, and the methylprednisolone. The single dose pill includes inactive ingredients including at least one of magnesium stearate, modified starch, powdered cellulose, pregelatinized starch, and sodium starch glycolate.

A preferred embodiment of a course of treatment is to take orally one single-dose pill per day for six days in the morning with food. The dosage in each of the pills is the following:

### Day 1

600 mg Paracetamol;

24 mg Lansoprazole;

24 mg Methylprednisolone

### Day 2

500 mg Paracetamol;

20 mg Lansoprazole;

20 mg Methylprednisolone

### Day 3

400 mg Paracetamol;

16 mg Lansoprazole;

16 mg Methylprednisolone

### Day 4

300 mg Paracetamol;

12 mg Lansoprazole;

12 mg Methylprednisolone

### Day 5

200 mg Paracetamol;

8 mg Lansoprazole;

8 mg Methylprednisolone

### Day 6

100 mg Paracetamol;

4 mg Lansoprazole;

4 mg Methylprednisolone

A study included twenty patients suffering from back and/or neck pain and administered the course of treatment. All the patients found the treatment to be effective to relieve the patients' pain without causing side effects. In particular, the patients reported no stomach pain during treatment. All the patients completed the entire course of treatment. All the patients confirmed taking the scheduled dosage at the scheduled time.

The results of the study contrast results when patients are given a course of doses broken into multiple pills containing smaller dosages of the active ingredients separately. When given multiple pills of separate ingredients, patients were found to take fewer-than-prescribed or even no doses of protein-pump inhibitor and paracetamol. Reasons given were complaints about the number of pills required or the perceived lack of need in light of improved symptoms. About thirty percent of patients who reported taking a steroid alone during a course of treatment complained of stomach pain. In turn, the stomach pain led to the patient abandoning the course of therapy and requesting alternatives. The alternative treatment prescribed is an epidural injection.

What is claimed is:

1. A treatment for inflammation and pain, comprising:
an anti-inflammatory;
an analgesic; and
a composition for reducing stomach acid.

2. The treatment according to claim 1, wherein said anti-inflammatory is a steroid.

3. The treatment according to claim 2, wherein said steroid is methylprednisolone.

4. The treatment according to claim 1, wherein said analgesic is paracetamol.

5. The treatment according to claim 1, wherein said composition for reducing stomach acid is a proton pump inhibitor.

6. The treatment according to claim 5, wherein said proton pump inhibitor is esomeprazole.

7. The treatment according to claim 5, wherein said proton pump inhibitor is lansoprazole.

8. The treatment according to claim 1, wherein said anti-inflammatory, said analgesic, and said composition for reducing stomach acid are included in a single dose.

9. The treatment according to claim 8, wherein:
said anti-inflammatory includes a therapeutic amount of methylprednisolone;
said analgesic includes a therapeutic amount of paracetamol; and
said composition for reducing stomach acid includes a therapeutic amount of lansoprazole.

10. A course of treatment for inflammation and pain, comprising:
a first dose to be given at a first time, said first dose including:
a therapeutic amount of methylprednisolone;
a therapeutic amount of paracetamol;
a therapeutic amount of lansoprazole; and
a delivery mechanism for controlling at least one of an amount and a duration of a delivery of one of said methylprednisolone, said paracetamol, and said lansoprazole so that when a second dose is given at a second time, said methylprednisolone, said paracetamol, and said lansoprazole all remain at a therapeutic level between said first time and said second time and at a safe level after said second time; and
said second dose to be given at said second time, said second dose including at least one of:
a therapeutic amount of methylprednisolone;
a therapeutic amount of paracetamol; and
a therapeutic amount of lansoprazole.

11. The treatment according to claim 1, wherein a dose of said anti-inflammatory, said analgesic, and said composition for reducing stomach acid are included in no more than one pill.

12. A method of treating inflammation and pain while reducing gastro-intestinal side effects, which comprises:
providing a therapeutic amount of an anti-inflammatory;
providing a therapeutic amount of an analgesic; and
providing a therapeutic amount of a stomach acid reducer.

13. The method according to claim 12, wherein said method is used to treat joint pain.

14. The method according to claim 13, wherein said method is used to treat back and neck pain.

15. The method according to claim 12, wherein said anti-inflammatory is a steroid.

16. The method according to claim 15, wherein said steroid is methylprednisolone.

17. The method according to claim 12, wherein said analgesic is paracetamol.

18. The method according to claim 12, wherein said stomach acid reducer is a proton pump inhibitor.

19. The method according to claim 18, wherein said proton pump inhibitor is esomeprazole.

20. The method according to claim 12, which further comprises delivering said anti-inflammatory, said analgesic, and said composition for reducing stomach acid to a patient in a single dose.

21. A method for treating inflammation and pain while reducing gastro-intestinal side effect over a course of treatment, which comprises:
delivering a first dose at a start of said course, said first dose including a therapeutically effective amount of an anti-inflammatory, a therapeutically effective amount of an analgesic, and a therapeutically effective amount of a composition for reducing stomach acid;
delivering a second dose at a second time during said course, said second dose including at least one of a therapeutically effective amount of an anti-inflammatory, a therapeutically effective amount of an analgesic, and a therapeutically effective amount of a composition for reducing stomach acid;
prolonging treatment of the inflammation and the pain while reducing the gastro-intestinal side effects between the start and the second time and while preventing exposure to unsafe amounts of said anti-inflammatories, said analgesics, and said compositions for reducing stomach acid by adjusting at least one of:
an amount of at least one of said anti-inflammatory, said analgesic, and said composition for reducing stomach acid in said first dose,
a delivery rate of at least one of said anti-inflammatory, said analgesic, and said composition for reducing stomach acid in said first dose;
an amount of at least one of said anti-inflammatory, said analgesic, and said composition for reducing stomach acid in said second dose, and
a delivery rate of at least one of said anti-inflammatory, said analgesic, and said composition for reducing stomach acid in said second dose.

22. The treatment according to claim 9, including:
between two and thirty-two milligrams of said methylprednisolone;
between one hundred and one thousand three hundred milligrams of said paracetamol; and
between two and thirty-two milligrams of said lansoprazole.

23. The course of treatment according to claim 10, wherein:
said first dose is given on a first day;
said second dose is given after said first dose; and
said second dose includes less of at least one of said methylprednisolone, said paracetamol, and said lansoprazole than said first dose.
